(19) United States
(12) Patent Application Publication
(10) Pub. No.: US 2022/0314975 A1
(43) Pub. Date: Oct. 6, 2022

(54) DRIVE ASSIST SYSTEM

(71) Applicant: SUBARU CORPORATION, Tokyo (JP)

(72) Inventors: Toru KATO, Tokyo (JP); Ryota NAKAMURA, Tokyo (JP); Tsukasa MIKUNI, Tokyo (JP); Masatoshi TSUGE, Tokyo (JP)

(21) Appl. No.: 17/693,926

(22) Filed: Mar. 14, 2022

(30) Foreign Application Priority Data
Mar. 30, 2021 (JP) 2021-058855

(51) Int. Cl. B60W 30/09; G07C 5/02; B60W 50/14; A61B 5/00; A61B 5/18; A61B 5/369; G06V 20/58

(52) U.S. Cl. CPC B60W 30/09; G07C 5/02; B60W 50/14; A61B 5/4809; A61B 5/18; A61B 5/369; G06V 20/582; B60W 2540/229; B60W 2540/221; B60W 2420/42; B60W 2555/60; B60W 2720/10; A61B 2503/22

(57) ABSTRACT

A drive assist system includes an occupant monitoring apparatus, an information acquisition apparatus, a determination apparatus, and a drive assist apparatus. The drive assist apparatus includes one or more processors, and one or more memories coupled to the one or more processors to communicate therewith. The one or more processors appropriately change a warning timing or a drive assist control intervention timing in accordance with the sleep state of a driver of a vehicle at least from a previous day, upon receiving information, from the determination apparatus, that the vehicle transitions from a stopped state to a traveling state. The warning timing is a timing of giving a warning that the vehicle is approaching another vehicle, or the like. The drive assist control intervention timing is a timing of intervention by the drive assist control for avoiding contact between the vehicle and the other vehicle or the like.

### CROSS-REFERENCE TO RELATED APPLICATIONS

The present application claims priority from Japanese Patent Application No. 2021-058855 filed on Mar. 30, 2021, the entire contents of which are hereby incorporated by reference.

### BACKGROUND

The technology relates to a drive assist system.

In general, at a driving start timing of a vehicle, it is difficult to determine a sleep state of an occupant, including a driver, inside the vehicle and to also determine the degree of the sleep state of the occupant.

Meanwhile, the following technique is disclosed regarding sleepiness of a driver. That is, a sleepiness level of the driver is detected, and whether awakening assistance for the driver is necessary is determined on the basis of the detected sleepiness level. In a case where the awakening assistance is necessary, a driver's seat is vibrated at least in some time slots with use of a vibration wave in which a first frequency promoting muscle tone and a second frequency suppressing muscle tone are imposed on each other. Such a technique is disclosed, for example, in Japanese Unexamined Patent Application Publication (JP-A) No. 2020-082906.

### SUMMARY

An aspect of the technology provides a drive assist system to be applied to the vehicle. The drive assist system includes an occupant monitoring apparatus, an information acquisition apparatus, a determination apparatus, and a drive assist apparatus. The occupant monitoring apparatus is configured to monitor a physical state of an occupant of the vehicle. The occupant includes a driver who drives the vehicle. The information acquisition apparatus is configured to acquire information including biometric information of the occupant when inside and outside the vehicle. The determination apparatus is configured to determine a traveling state and a stopped state of the vehicle. The drive assist apparatus is configured to determine a sleep state of the occupant on the basis of monitoring information acquired by the occupant monitoring apparatus, acquisition information acquired by the information acquisition apparatus, or both, and execute a drive assist control for securing safe traveling of the vehicle. The sleep state includes sleep duration and quality of sleep. The drive assist apparatus includes one or more processors, and one or more memories. The one or more memories are coupled to the one or more processors to communicate with the one or more processors. The one or more processors are configured to appropriately change a warning timing or a drive assist control intervention timing in accordance with the sleep state of the driver at least from a previous day, upon receiving information, from the determination apparatus, that the vehicle transitions from the stopped state to the traveling state. The warning timing is a timing of giving a warning that the vehicle is approaching another vehicle, an object, a human, or an animal. The drive assist control intervention timing is a timing of intervention by the drive assist control for avoiding contact between the vehicle and the other vehicle, the object, the human, or the animal.

### BRIEF DESCRIPTION OF THE DRAWINGS

The accompanying drawings are included to provide a further understanding of the disclosure, and are incorporated in and constitute a part of this specification. The drawings illustrate embodiments and, together with the specification, serve to explain the principles of the disclosure.

FIG. 1 is a diagram illustrating an example of a configuration of a drive assist system according to an example embodiment of the technology.

FIG. 2 is a diagram illustrating an example of a configuration of a drive control apparatus of the drive assist system according to the example embodiment of the technology.

FIG. 3 is a diagram illustrating an example of a concept used in a case where the drive control apparatus of the drive assist system according to the example embodiment of the technology sets a predetermined time.

FIG. 4 is a flowchart illustrating an example of a process to be performed by the drive assist system according to the example embodiment of the technology.

FIG. 5 is a diagram illustrating an example of a configuration of a drive assist system according to an example embodiment of the technology.

FIG. 6 is a diagram illustrating an example of a configuration of a drive control apparatus of the drive assist system according to the example embodiment of the technology.

FIG. 7 is a flowchart illustrating an example of a process to be performed by the drive assist system according to the example embodiment of the technology.

FIG. 8 is a diagram illustrating an example of a configuration of a drive assist system according to an example embodiment of the technology.

FIG. 9 is a diagram illustrating an example of a configuration of a drive control apparatus of the drive assist system according to the example embodiment of the technology.

FIG. 10 is a flowchart illustrating an example of a process to be performed by the drive assist system according to the example embodiment of the technology.

FIG. 11 is a diagram illustrating an example of a configuration of a drive assist system according to an example embodiment of the technology.

FIG. 12 is a diagram illustrating an example of a configuration of a drive control apparatus of the drive assist system according to the example embodiment of the technology.

FIG. 13 is a flowchart illustrating an example of a process to be performed by the drive assist system according to the example embodiment of the technology.

FIG. 14 is a diagram illustrating an example of a configuration of a drive assist system according to an example embodiment of the technology.

FIG. 15 is a diagram illustrating an example of a configuration of a drive control apparatus of the drive assist system according to the example embodiment of the technology.

FIG. 16 is a flowchart illustrating an example of a process to be performed by the drive assist system according to the example embodiment of the technology.

FIG. 17 is a diagram illustrating an example of a configuration of a drive assist system according to an example embodiment of the technology.

FIG. 18 is a diagram illustrating an example of a configuration of a drive control apparatus of the drive assist system according to the example embodiment of the technology.

FIG. 19 is a flowchart illustrating an example of a process to be performed by the drive assist system according to the example embodiment of the technology.

### DETAILED DESCRIPTION

The technique disclosed in JP-A No. 2020-082906 is merely for determining a sleepiness level of a driver of a vehicle in the middle of traveling of the vehicle. Therefore, it is difficult to determine a sleep state of an occupant, including the driver, inside the vehicle and perform drive assistance for safe traveling of the vehicle at a driving start timing of the vehicle.

It is desirable to provide a drive assist system that determines a sleep state of an occupant, including a driver, inside a vehicle and performs appropriate drive assistance for safe traveling of the vehicle at a driving start timing of the vehicle.

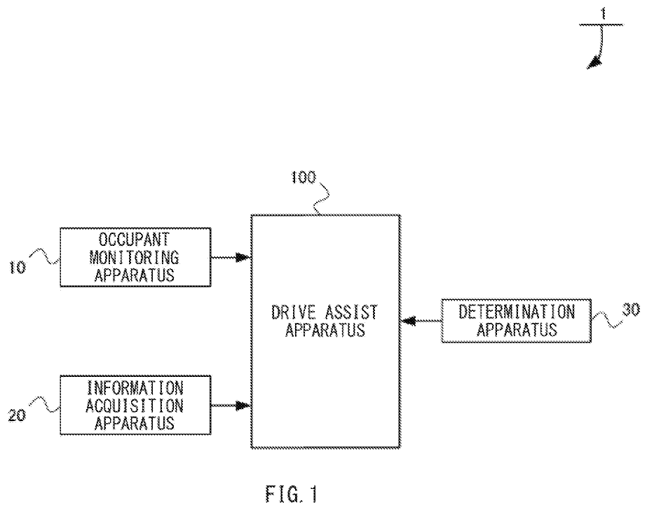

In the following, some example embodiments of the technology are described with reference to FIGS. 1 to 19. Note that the following description is directed to illustrative examples of the disclosure and not to be construed as limiting to the technology. In each of the drawings referred to in the following description, elements have different scales in order to illustrate the respective elements with sizes recognizable in the drawings. Therefore, factors including, without limitation, the number of each of the elements, the shape of each of the elements, a size of each of the elements, a ratio between the elements, and relative positional relationship between the elements are illustrative only and not to be construed as limiting to the technology. Further, elements in the following example embodiments which are not recited in a most-generic independent claim of the disclosure are optional and may be provided on an as-needed basis. Throughout the present specification and the drawings, elements having substantially the same function and configuration are denoted with the same numerals to avoid any redundant description.

### First Example Embodiment

Referring to FIGS. 1 to 4, a drive assist system 1 according to a first example embodiment of the technology is described.

### [Configuration of Drive Assist System 1]

As illustrated in FIG. 1, the drive assist system 1 according to the first example embodiment may include an occupant monitoring apparatus 10, an information acquisition apparatus 20, a determination apparatus 30, and a drive assist apparatus 100.

The occupant monitoring apparatus 10 monitors a physical state of an occupant of a vehicle. As used herein, the term “occupant” encompasses a driver. Non-limiting examples of the physical state of the occupant of the vehicle may include a behavior, a facial expression, and a health condition of the occupant of the vehicle.

The occupant monitoring apparatus 10 may include, for example, a built-in imaging device such as a charge coupled device (CCD) or a complementary metal oxide semiconductor (CMOS) image sensor (CIS). The occupant monitoring apparatus 10 may monitor the physical state or the like of the occupant of the vehicle with use of a camera. The camera may acquire an image, such as a moving image or a still image, of the occupant of the vehicle captured by the imaging device.

In one example embodiment, the camera may include both an optical camera and a near-infrared ray camera.

Non-limiting examples of information to be acquired from the occupant monitoring apparatus 10 may include an opening degree of an eye, the number of times of blinking, sleep duration, rest duration, and the number of times of rolling while sleeping.

The information acquisition apparatus 20 may be provided inside or outside of the vehicle. The information acquisition apparatus 20 acquires, for example, biometric information of the occupant. Non-limiting examples of the biometric information may include vital data of each occupant.

Non-limiting examples of the information acquisition apparatus 20 provided inside the vehicle may include an apparatus mounted on the vehicle and an apparatus worn or carried by the occupant, such as a smartwatch or a smartphone.

Non-limiting examples of the information acquisition apparatus 20 provided outside the vehicle may include a fixed apparatus and a movable apparatus provided in the occupant's home. Non-limiting examples of the fixed apparatus may include an apparatus provided on a ceiling or a wall of a bedroom, a stationary apparatus, and an apparatus embedded in a bed mattress. Non-limiting examples of the movable apparatus may include an apparatus mounted on a robot.

Non-limiting examples of information to be obtained from the information acquisition apparatus 20 may include a heart rate, blood-oxygen saturation, heart rate variability, a respiratory rate, a body temperature, a blood pressure, a hemoglobin level, an activity amount, the number of times of rolling while sleeping, a sleep state, and sleep duration.

Here, for example but not limited to, the number of times of rolling while sleeping, a REM sleep state, and a non-REM state may serve as example indices used for detecting the sleep state including quality of sleep.

The determination apparatus 30 determines a traveling state and a stopped state of the vehicle. The determination apparatus 30 may include, for example but not limited to, an ignition switch that determines a state of an ignition key. For example, in a case where the ignition key is at an ON position, the determination apparatus 30 may determine that the vehicle is in the traveling state. In a case where the ignition key is at an OFF position, the determination apparatus 30 may determine that the vehicle is in the stopped state.

In one example, the determination apparatus 30 may be mounted on the vehicle and include a seat device configured to freely change an angle of a seat back. The determination apparatus 30 may determine the traveling state or the stopped state of the vehicle on the basis of an operation angle of the seat back, i.e., an angle of the seat back set by the driver's operation.

In this case, the determination apparatus 30 may include, for example but not limited to, a sensor detecting the operation angle of the seat back. In a case where the operation angle of the seat back is a predetermined angle or greater, the determination apparatus 30 may determine that the vehicle is in the stopped state. The “predetermined angle” may be, for example but not limited to, an operation angle of the seat back in a case where the driver takes a rest.

Note that, the term “traveling state” encompasses not only a state in which the vehicle is actually traveling but also a state in which the vehicle is ready to travel.

The drive assist apparatus 100 determines a sleep state on the basis of monitoring information acquired by the occupant monitoring apparatus 10, acquisition information acquired by the information acquisition apparatus 20, or both, and executes a drive assist control for securing safe traveling of the vehicle. The sleep state may include the sleep duration and the quality of sleep.

The drive assist apparatus 100 appropriately changes: a timing of giving a warning that the vehicle is approaching another vehicle, an object, a human, or an animal; or a timing of intervention by the drive assist control for avoiding contact between the vehicle and another vehicle, an object, a human, or an animal, in accordance with the sleep state of the driver among the occupants of the vehicle at least from a previous day, upon receiving information, from the determination apparatus 30, that the vehicle transitions from the stopped state to the traveling state.

The drive assist apparatus 100 may refrain from changing the timing of giving the warning that the vehicle is approaching another vehicle, an object, a human, or an animal or the timing of intervention by the drive assist control for avoiding contact between the vehicle and another vehicle, an object, a human, or an animal, in a case where the accumulated sleep duration of the driver at least from the previous day is a predetermined time or more.

The drive assist apparatus 100 may refrain from changing the timing of giving the warning that the vehicle is approaching another vehicle, an object, a human, or an animal or the timing of intervention by the drive assist control for avoiding contact between the vehicle and another vehicle, an object, a human, or an animal, in a case where the quality of sleep of the driver is a predetermined level or higher.

The configuration of the drive assist apparatus 100 and the process to be performed by the drive assist apparatus 100 may be described in detail later.

### [Configuration of Drive Assist Apparatus 100]

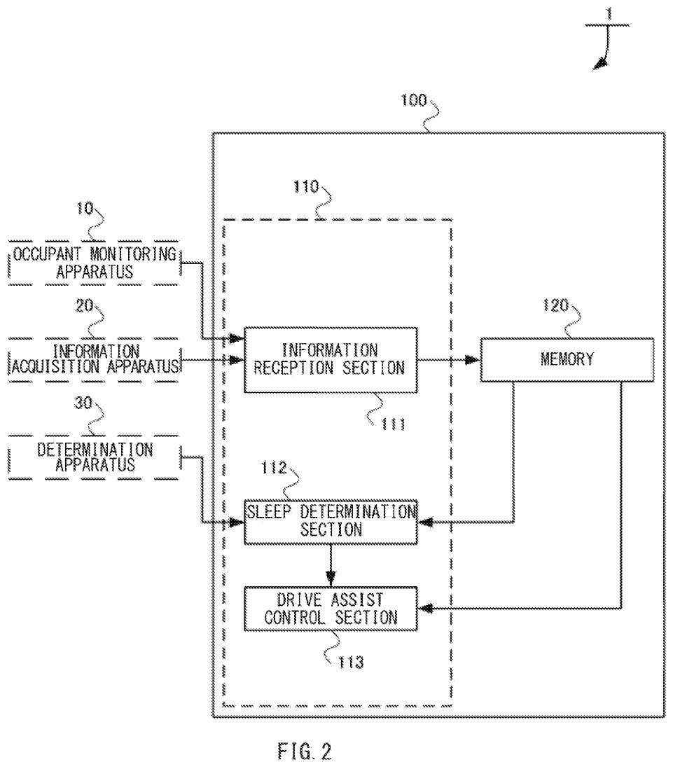

As illustrated in FIG. 2, the drive assist apparatus 100 according to the first example embodiment includes a processor 110 and a memory 120.

The processor 110 may perform a control of the drive assist apparatus 100 as a whole in accordance with a control program. The control program may be stored in the memory 120 which will be described later.

According to the first example embodiment, the processor 110 may execute processes to be performed by sections including, without limitation, an information reception section 111, a sleep determination section 112, and a drive assist control section 113 which will be described later.

The memory 120 may include, for example but not limited to, a read-only memory (ROM) and a random-access memory (RAM). The ROM may have, for example but not limited to, the above-described control program stored therein. The RAM may have, for example but not limited to, various pieces of data stored therein.

According to the first example embodiment, for example, information from the occupant monitoring apparatus 10, the information acquisition apparatus 20, and any other suitable information may be stored in the RAM.

### [Configuration of Processor 110]

As illustrated in FIG. 2, the processor 110 may include the information reception section 111, the sleep determination section 112, and the drive assist control section 113.

The information reception section 111 may receive, from the determination apparatus 30, information that the vehicle is in the traveling state or information that the vehicle is in the stopped state.

Further, the information reception section 111 may receive, from the occupant monitoring apparatus 10, information regarding the physical state of the occupant of the vehicle including, without limitation, the behavior, the facial expression, and the health condition of the occupant. In the first example embodiment, the information reception section 111 may receive information regarding the sleep state including, without limitation, the sleep duration and the quality of sleep of the driver.

Further, the information reception section 111 may receive, from the information acquisition apparatus 20, information of, for example but not limited to, the heart rate, the blood-oxygen saturation, the heart rate variability, the respiratory rate, the body temperature, the blood pressure, the hemoglobin level, the activity amount, the number of times of rolling while sleeping, the sleep state, and the sleep duration. In the first example embodiment, the information reception section 111 may receive information regarding the sleep state including, without limitation, the sleep duration and the quality of sleep of the driver.

The information reception section 111 may output the information received from the occupant monitoring apparatus and the information received from the information acquisition apparatus 20 to the RAM in the memory 120.

Upon receiving, from the determination apparatus 30, information that the vehicle has transitioned from the stopped state to the traveling state, the sleep determination section 112 may determine, for example, the sleep state including, without limitation, the sleep duration and the quality of sleep of the driver on the basis of the monitoring information acquired by the occupant monitoring apparatus 10 and stored in the RAM in the memory 120, the acquisition information acquired by the information acquisition apparatus 20, or both.

For example, in a case where the sleep duration (accumulated sleep duration) is mainly used as an index of determination of the sleep state, the sleep determination section 112 may determine the sleep state on the basis of whether the sleep duration in the acquired information is longer than a predetermined time.

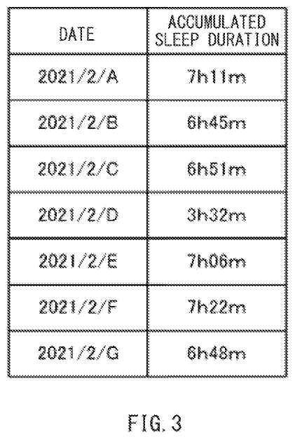

For example, the above-mentioned “predetermined time” may be set from accumulated sleep duration of each day measured for each driver as described in FIG. 3, on the basis of the monitoring information acquired by the occupant monitoring apparatus 10 for a week, the acquisition information acquired by the information acquisition apparatus 20 for a week, or both.

In one example, referring to an example in FIG. 3, “predetermined time” may be set from an average value of values of the accumulated sleep duration of respective days measured for each driver. In another example, the “predetermined time” may be set as a peak time in a distribution of the accumulated sleep duration of each day measured for each driver.

Note that, although FIG. 3 presents an example only for a particular person, similar data may need to be prepared for other occupants who may possibly drive the vehicle.

In a case where the quality of sleep is mainly used as the index of the determination of the sleep state, the sleep determination section 112 may determine the sleep state on the basis of whether the quality of sleep of the driver is at a predetermined level or higher. The information regarding the level of the quality of sleep of the driver may be acquired from the acquired information regarding the quality of sleep such as the number of times of rolling while sleeping, the REM sleep state, or the non-REM state.

The above-mentioned “predetermined level” may be determined using information already known as knowledge as a reference.

The drive assist control section 113 may execute the drive assist control for securing the safe traveling of the vehicle on the basis of a determination result obtained by the sleep determination section 112.

Further, the drive assist control section 113 may appropriately change the timing of giving the warning that the vehicle is approaching another vehicle, an object, a human, or an animal, or the timing of the intervention by the drive assist control for avoiding the contact between the vehicle and another vehicle, an object, a human, or an animal, in accordance with the sleep state of the driver among the occupants of the vehicle at least from the previous day, upon receiving information, from the determination apparatus 30, that the vehicle transitions from the stopped state to the traveling state.

Note that the drive assist control section 113 may change the drive assist control intervention timing by rewriting an initial value stored in the RAM in the memory 120 to a changed value.

For example, in a case where the accumulated sleep duration of the driver at least from the previous day is the predetermined time or more, the drive assist control section 113 may refrain from changing the timing of giving the warning that the vehicle is approaching another vehicle, an object, a human, or an animal, or the timing of the intervention by the drive assist control for avoiding the contact between the vehicle and another vehicle, an object, a human, or an animal.

For example, in a case where the quality of sleep of the driver is the predetermined level or higher, the drive assist control section 113 may refrain from changing the timing of giving the warning that the vehicle is approaching another vehicle, an object, a human, or an animal, or the timing of the intervention by the drive assist control for avoiding the contact between the vehicle and another vehicle, an object, a human, or an animal.

Non-limiting examples of the drive assist control may include changing of the timing of giving the warning that the vehicle is approaching another external vehicle, an object, a human, or an animal, or the timing of the intervention by the drive assist control for avoiding contact.

More specific but non-limiting examples of the drive assist control may include: a control of changing a notification threshold of a sleeping detection warning based on a driver monitoring system (DMS) and making a timing of notification of the sleeping detection warning earlier; a control of automatically setting a warning inter-vehicle distance to the maximum distance to perform earlier warning of rear end contact; a control of forcefully turning on an LED light or outputting a warning sound in a rear and side alert assist system; a control of forcefully causing a lane departure warning (LDW) system to be in an ON mode; a control of forcefully causing an automated lane-keeping system (ALKS) to be in an ON mode; a control of lowering a set upper limit in vehicle speed setting in a cruise control; a control of making an automatic emergency steering (AES) intervention timing earlier; and a control of making an autonomous emergency braking (AEB) intervention timing earlier.

### [Process of Drive Assist Apparatus 100]

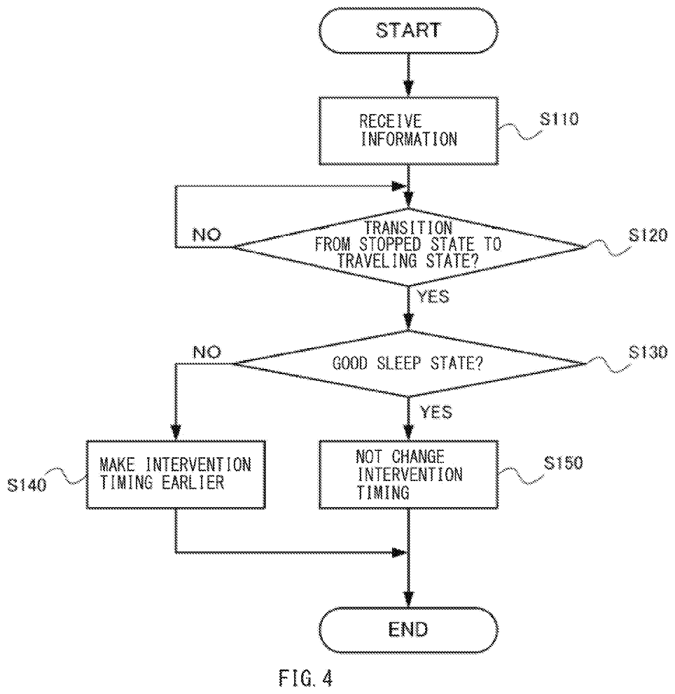

Referring to FIG. 4, a process to be performed by the drive assist apparatus 100 according to the first example embodiment is described.

The information reception section 111 of the drive assist apparatus 100 may receive information from the occupant monitoring apparatus 10 and information from the information acquisition apparatus 20 (step S110).

Further, the information reception section 111 may output the information received from the occupant monitoring apparatus 10 and the information acquisition apparatus 20 to the RAM in the memory 120.

The sleep determination section 112 of the drive assist apparatus 100 may determine whether the sleep determination section 112 has received, from the determination apparatus 30, information that the vehicle has transitioned from the stopped state to the traveling state (step S120). Further, in a case where the sleep determination section 112 determines that the sleep determination section 112 has not received, from the determination apparatus 30, the information that the vehicle has transitioned from the stopped state to the traveling state (NO in step S120), the process may be caused to return to step S120, and the drive assist apparatus 100 may transition to a standby mode.

In a case where the sleep determination section 112 determines that the sleep determination section 112 has received, from the determination apparatus 30, the information that the vehicle has transitioned from the stopped state to the traveling state (YES in step S120), the sleep determination section 112 may acquire information for determining the sleep state including the sleep duration and the quality of sleep of the driver from the information stored in the RAM in the memory 120, and determine whether the sleep state of the driver is good (step S130).

In a case where the sleep determination section 112 determines that the sleep state of the driver is good (YES in step S130), the sleep determination section 112 may supply the information that the sleep state of the driver is good to the drive assist control section 113. Further, the drive assist control section 113, which has received the information from the sleep determination section 112, may maintain the drive assist control invention timing as it is (step S150).

In a case where the sleep determination section 112 determines that the sleep state of the driver is not good (NO in step S130), the sleep determination section 112 may supply the information that the sleep state of the driver is not good to the drive assist control section 113. Further, the drive assist control section 113, which has received the information from the sleep determination section 112, may make the drive assist control invention timing earlier than as it is (step S140).

### Example Workings and Example Effects

As described above, in the drive assist system 1 according to the first example embodiment, the processor 110 of the drive assist apparatus 100 may appropriately change the timing of giving the warning that the vehicle is approaching another vehicle, an object, a human, or an animal, or the timing of the intervention by the drive assist control for avoiding the contact between the vehicle and another vehicle, an object, a human, or an animal, in accordance with the sleep state of the driver among the occupants of the vehicle at least from the previous day, upon receiving information, from the determination apparatus 30, that the vehicle transitions from the stopped state to the traveling state.

That is, in a case where the sleep state of the driver is not good, it is highly possible that safe traveling of the vehicle is disturbed. To address this, the processor 110 of the drive assist apparatus 100 may determine the sleep state of the driver at least from the previous day. In a case where the sleep state of the driver is not good, the processor 110 may appropriately change the timing of the intervention by the drive assist control.

As a result, it is possible to perform appropriate drive assistance for safe traveling.

Further, in a case where the accumulated sleep duration of the driver at least from the previous day is the predetermined time or more, the processor 110 of the drive assist apparatus 100 may refrain from changing the timing of giving the warning that the vehicle is approaching another vehicle, an object, a human, or an animal or the timing of the intervention by the drive assist control for avoiding the contact between the vehicle and another vehicle, an object, a human, or an animal.

That is, in a case where the accumulated sleep duration of the driver at least from the previous day is the predetermined time or more, the sleep state of the driver can be considered good. Therefore, in such a case, the processor 110 of the drive assist apparatus 100 may refrain from changing the timing of the intervention by the drive assist control.

As a result, it is possible to perform appropriate drive assistance for safe traveling.

Further, the processor 110 of the drive assist apparatus 100 may set the predetermined time from the accumulated sleep duration of each day measured for each driver, on the basis of the monitoring information acquired by the occupant monitoring apparatus 10, the acquisition information acquired by the information acquisition apparatus 20, or both.

This makes it possible to determine the sleep state of the driver using, as a reference, the predetermined time for each occupant who drives the vehicle, allowing for highly accurate determination.

As a result, it is possible to perform appropriate drive assistance for safe traveling.

Further, in a case where the quality of sleep of the driver is the predetermined level or higher, the processor 110 of the drive assist apparatus 100 may refrain from changing the timing of giving the warning that the vehicle is approaching another vehicle, an object, a human, or an animal or the timing of the intervention by the drive assist control for avoiding the contact between the vehicle and another vehicle, an object, a human, or an animal.

That is, in a case where the quality of sleep of the driver is the predetermined level or higher, the sleep state of the driver can be considered good. Therefore, in such a case, the processor 110 of the drive assist apparatus 100 may refrain from changing the timing of the intervention by the drive assist control.

As a result, it is possible to perform appropriate drive assistance for safe traveling.

Further, in a case where the occupant monitoring apparatus 10 includes both the optical camera and the near-infrared ray camera, it is possible to acquire an image of the occupant at any time of day and night.

This makes it possible to precisely acquire information regarding the sleep state of the occupant. As a result, it is possible to perform appropriate drive assistance.

### Second Example Embodiment

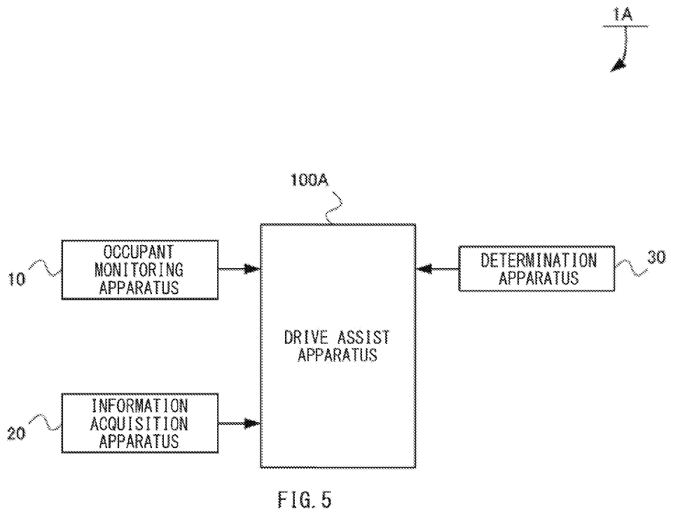

Referring to FIGS. 5 to 7, a drive assist system 1A according to a second example embodiment of the technology is described.

### [Configuration of Drive Assist System 1A]

As illustrated in FIG. 5, the drive assist system 1A according to the second example embodiment may include the occupant monitoring apparatus 10, the information acquisition apparatus 20, the determination apparatus 30, and a drive assist apparatus 100A.

Note that the components denoted with the same referential signs as those in the first example embodiment may serve as the same, and are therefore not described further in detail here.

The drive assist apparatus 100A may determine an awareness level of the driver on the basis of the monitoring information acquired by the occupant monitoring apparatus 10, the acquisition information acquired by the information acquisition apparatus 20, or both. In a case where the awareness level of the driver is determined as being low, the drive assist apparatus 100A may increase the level of the drive assist control for securing the safe traveling of the vehicle.

Details will be described later.

### [Configuration of Drive Assist Apparatus 100A]

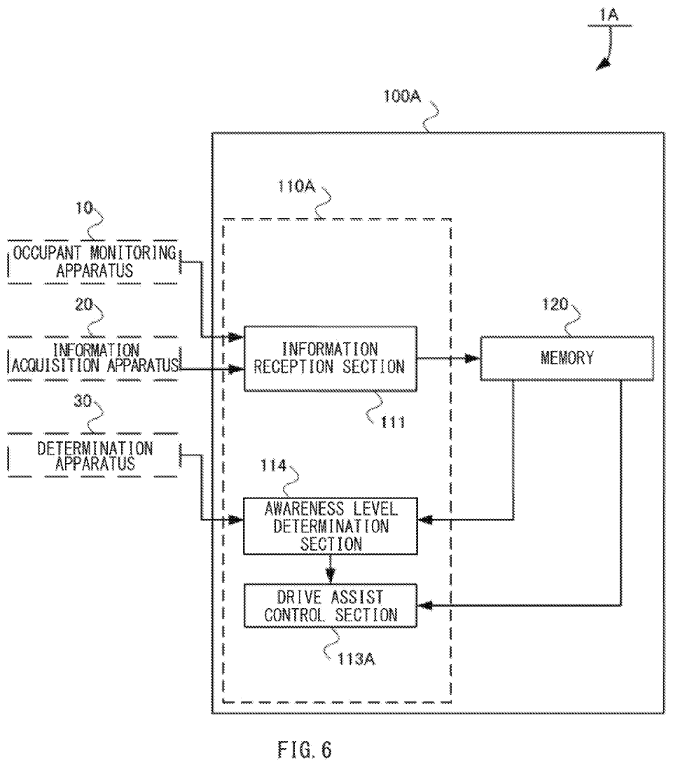

As illustrated in FIG. 6, the drive assist apparatus 100A may include a processor 110A and the memory 120. The processor 110A will be described in detail later.

### [Configuration of Processor 110A]

As illustrated in FIG. 6, the processor 110A may include the information reception section 111, a drive assist control section 113A, and an awareness level determination section 114.

In a case where the later-described awareness level determination section 114 determines that the awareness level of the driver is low on the basis of the monitoring information acquired by the occupant monitoring apparatus 10, the acquisition information acquired by the information acquisition apparatus 20, or both, the drive assist control section 113A may increase the level of the drive assist control for securing the safe traveling of the vehicle.

Note that the drive assist control section 113A may change the level of the drive assist control by rewriting an initial value stored in the RAM in the memory 120 to a changed value.

The awareness level determination section 114 may determine the awareness level of the driver from the sleep start time and the sleep duration of the driver at least of the previous night on the basis of the monitoring information acquired by the occupant monitoring apparatus 10, the acquisition information acquired by the information acquisition apparatus 20, or both.

The awareness level determination section 114 may also determine the awareness level of the driver from variation in brain waves of the driver on the basis of the acquisition information acquired by the information acquisition apparatus 20.

### [Process of Drive Assist Apparatus 100A]

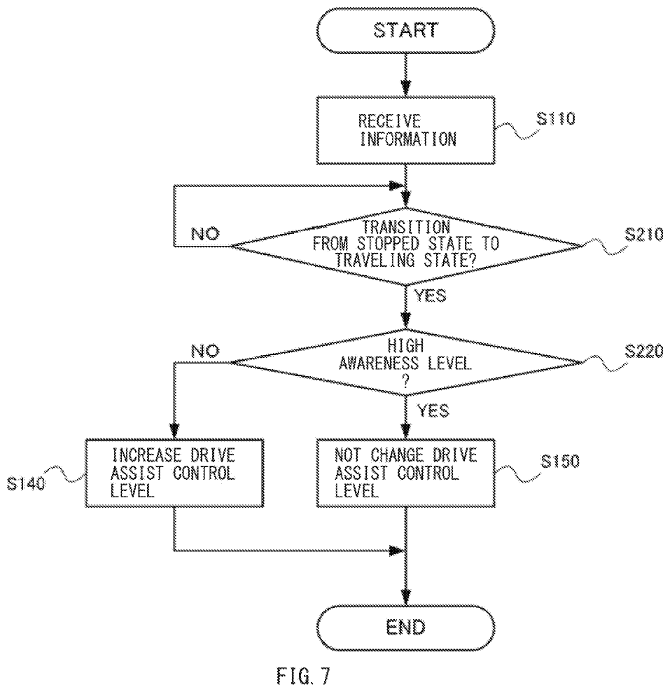

Referring to FIG. 7, a process to be performed by the drive assist apparatus 100A according to the second example embodiment is described.

The information reception section 111 of the drive assist apparatus 100A may receive information from the occupant monitoring apparatus 10 and information from the information acquisition apparatus 20 (step S110).

Further, the information reception section 111 may output the information received from the occupant monitoring apparatus 10 and the information acquisition apparatus 20 to the RAM in the memory 120.

The awareness level determination section 114 of the drive assist apparatus 100A may determine whether the awareness level determination section 114 has received, from the determination apparatus 30, information that the vehicle has transitioned from the stopped state to the traveling state (step S210). Further, in a case where the awareness level determination section 114 determines that the awareness level determination section 114 has not received, from the determination apparatus 30, the information that the vehicle has transitioned from the stopped state to the traveling state (NO in step S210), the process may be caused to return to step S210, and the drive assist apparatus 100A may transition to a standby mode.

In a case where the awareness level determination section 114 determines that the awareness level determination section 114 has received, from the determination apparatus 30, the information that the vehicle has transitioned from the stopped state to the traveling state (YES in step S210), the awareness level determination section 114 may acquire, for example, information for determining the awareness level from the information stored in the RAM in the memory 120, and determine whether the awareness level of the driver is high (step S220).

In a case where the awareness level determination section 114 determines that the awareness level of the driver is high (YES in step S220), the awareness level determination section 114 may supply the information that the awareness level of the driver is high to the drive assist control section 113A. Further, the drive assist control section 113A, which has received the information from the awareness level determination section 114, may maintain the level of the drive assist control as it is (step S150).

In a case where the awareness level determination section 114 determines that the awareness level of the driver is low (NO in step S220), the awareness level determination section 114 may supply the information that the awareness level of the driver is low to the drive assist control section 113A. Further, the drive assist control section 113A, which has received the information from the awareness level determination section 114, may increase the level of the drive assist control (step S140).

### Example Workings and Example Effects

As described above, the processor 110A of the drive assist apparatus 100A in the drive assist system 1A according to the second example embodiment may determine the awareness level of the driver on the basis of the monitoring information acquired by the occupant monitoring apparatus 10, the acquisition information acquired by the information acquisition apparatus 20, or both. In a case where the awareness level of the driver is determined as being low, the processor 110A may increase the level of the drive assist control for securing the safe traveling of the vehicle.

That is, in a case of driving of the vehicle by a driver having a low awareness level, it can be considered that the determination ability at the time of traveling is also low.

Therefore, in the case where the awareness level of the driver is low, increasing of the level of the drive assist control for securing the safe traveling of the vehicle makes it possible to perform appropriate drive assistance for safe traveling.

Further, the processor 110A of the drive assist apparatus 100A may determine the awareness level of the driver from the sleep start time and the sleep duration of the driver at least of the previous night on the basis of the monitoring information acquired by the occupant monitoring apparatus 10, the acquisition information acquired by the information acquisition apparatus 20, or both.

That is, a basic physical function of a human works normally with a circadian rhythm. This rhythm is created by a biological clock at the suprachiasmatic nucleus situated in the hypothalamus. A human determines day and night environments and adjusts the biological function on the basis of this rhythm. A lack of sleep or an irregular lifestyle may disturb the biological rhythm created in the brain, which greatly influences the awareness state.

Therefore, determining the awareness level of the driver from the sleep start time and the sleep duration of the driver at least of the previous night makes it possible to accurately determine the awareness level of the driver.

Further, the processor 110A of the drive assist apparatus 100A may determine the awareness level of the driver from the variation in brain waves of the driver on the basis of the acquisition information acquired by the information acquisition apparatus 20.

That is, it has been found that a decrease in awareness level causes variation in a waves, more specifically, variation in distribution or a decrease in frequency of the a waves.

Therefore, determining the awareness level of the driver from the variation in brain waves acquired from the information acquisition apparatus 20 makes it possible to accurately determine the awareness level of the driver.

### Third Example Embodiment

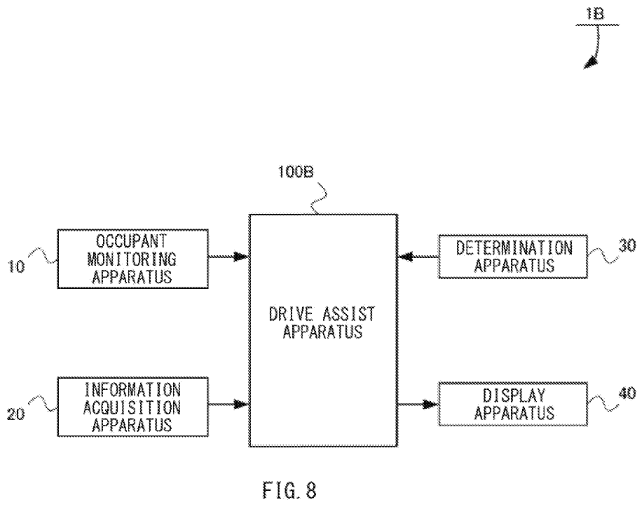

Referring to FIGS. 8 to 10, a drive assist system 1B according to a third example embodiment of the technology is described.

### [Configuration of Drive Assist System 1B]

As illustrated in FIG. 8, the drive assist system 1B according to the third example embodiment may include the occupant monitoring apparatus 10, the information acquisition apparatus 20, the determination apparatus 30, a display apparatus 40, and a drive assist apparatus 100B.

Note that the components denoted with the same referential signs as those in the first and the second example embodiments may serve as the same, and are therefore not described further in detail here.

The display apparatus 40 may display an image prompting the driver to perform an action of visually checking the front, the rear, the left, and the right of the vehicle.

The display apparatus 40 may be provided at four locations, e.g., at front, rear, left, and right positions, in a vehicle compartment. In one example, the display apparatus 40 may display the image prompting the driver to perform the action of visually checking the front, the rear, the left, and the right of the vehicle on a monitor of the occupant monitoring apparatus 10. In another example, the display apparatus 40 may add an audio guidance to such an image to guide the driver.

The drive assist apparatus 100B may cause the display apparatus 40 to display the image prompting the driver to perform the action of visually checking the front, the rear, the left, and the right of the vehicle.

Further, the drive assist apparatus 100B may observe the driver's behavior on the basis of the information obtained from the occupant monitoring apparatus 10. In a case where the driver does not appropriately perform visual checking, the drive assist apparatus 100B may perform a control of prohibiting starting of the vehicle.

### [Configuration of Drive Assist Apparatus 100B]

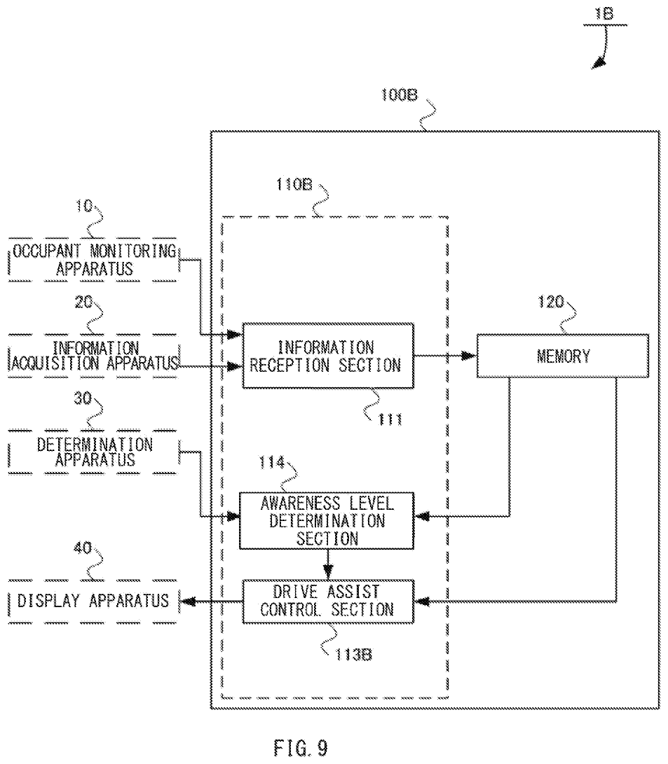

As illustrated in FIG. 9, the drive assist apparatus 100B may include a processor 110B and the memory 120. The processor 110B will be described in detail later.

### [Configuration of Processor 110B]

As illustrated in FIG. 9, the processor 110B may include the information reception section 111, a drive assist control section 113B, and the awareness level determination section 114.

The drive assist control section 113B may cause the display apparatus 40 to display the image prompting the driver to perform the action of visually checking the front, the rear, the left, and the right of the vehicle.

Further, the drive assist control section 113B may read the information acquired from the occupant monitoring apparatus 10 from the RAM in the memory 120 in which the information obtained from the occupant monitoring apparatus is stored. The drive assist control section 113B may observe the driver's behavior. In a case where the driver does not appropriately perform visual checking, the drive assist control section 113B may execute a control of prohibiting starting of the vehicle.

### [Process of Drive Assist Apparatus 100B]

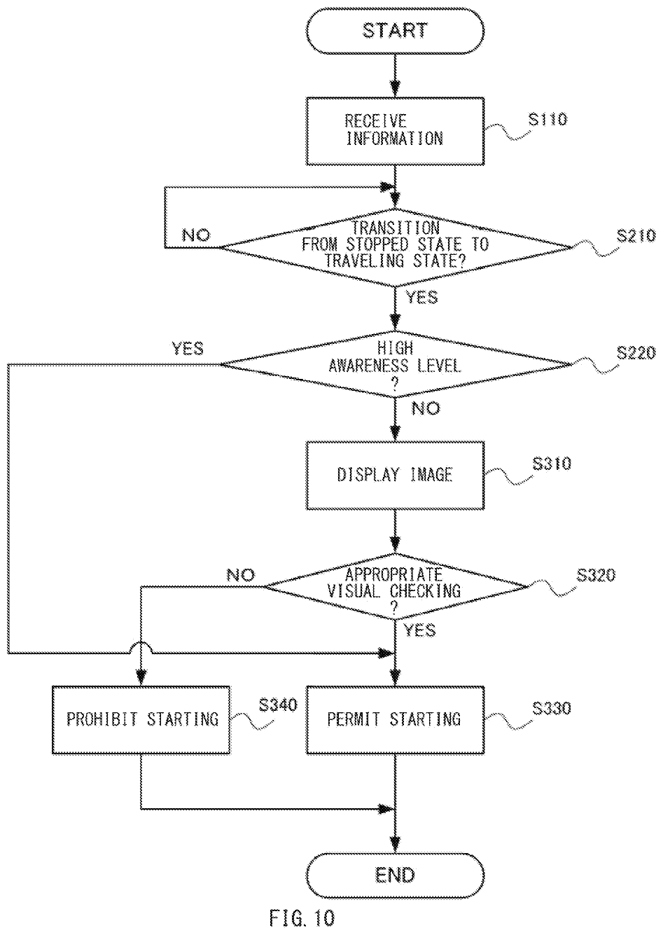

Referring to FIG. 10, a process to be performed by the drive assist apparatus 100B according to the third example embodiment is described.

The information reception section 111 of the drive assist apparatus 100B may receive information from the occupant monitoring apparatus 10 and information from the information acquisition apparatus 20 (step S110).

Further, the information reception section 111 may output the information received from the occupant monitoring apparatus 10 and the information acquisition apparatus 20 to the RAM in the memory 120.

The awareness level determination section 114 of the drive assist apparatus 100B may determine whether the awareness level determination section 114 has received, from the determination apparatus 30, information that the vehicle has transitioned from the stopped state to the traveling state (step S210). Further, in a case where the awareness level determination section 114 determines that the awareness level determination section 114 has not received, from the determination apparatus 30, the information that the vehicle has transitioned from the stopped state to the traveling state (NO in step S210), the process may be caused to return to step S210, and the drive assist apparatus 100B may transition to a standby mode.

In a case where the awareness level determination section 114 determines that the awareness level determination section 114 has received, from the determination apparatus 30, the information that the vehicle has transitioned from the stopped state to the traveling state (YES in step S210), the awareness level determination section 114 may acquire, for example, information for determining the awareness level from the information stored in the RAM in the memory 120, and determine whether the awareness level of the driver is high (step S220).

In a case where the awareness level determination section 114 determines that the awareness level of the driver is high (YES in step S220), the awareness level determination section 114 may supply the information that the awareness level of the driver is high to the drive assist control section 113B. Further, the drive assist control section 113B, which has received the information from the awareness level determination section 114, may cause the process to move to step S330.

In a case where the awareness level determination section 114 determines that the awareness level of the driver is low (NO in step S220), the awareness level determination section 114 may supply the information that the awareness level of the driver is low to the drive assist control section 113B. Further, the drive assist control section 113B, which has received the information from the awareness level determination section 114, may cause the display apparatus 40 to display the image prompting the driver to perform the action of visually checking the front, the rear, the left, and the right of the vehicle (step S310).

The drive assist control section 113B may read the information acquired by the occupant monitoring apparatus 10 from the RAM in the memory 120 in which the information obtained from the occupant monitoring apparatus 10 is stored, observe the driver's behavior, and determine whether the driver appropriately performs the visual checking (step S320).

In a case where the driver is determined as appropriately performing the visual checking (YES in step S320), and where the awareness level of the driver is determined as being high, the drive assist control section 113B may permit the starting of the vehicle (step S330), and end the process.

In a case where the driver is determined as not appropriately performing the visual checking (NO in step S320), the drive assist control section 113B may prohibit the starting of the vehicle (step S340), and end the process.

### Example Workings and Example Effects

As described above, in the drive assist system 1B according to the third example embodiment, in a case where the awareness level determination section 114 determines that the awareness level of the driver is low, the drive assist control section 113B may prompt the driver to perform the action of visually checking the front, the rear, the left, and the right of the vehicle before the vehicle starts traveling. Further, the drive assist control section 113B may observe the driver's behavior on the basis of the information obtained from the occupant monitoring apparatus 10. In a case where the driver does not appropriately perform the visual checking, the drive assist control section 113B may perform a control of prohibiting the starting of the vehicle.

That is, in a case of driving of the vehicle by a driver having a low awareness level, it can be considered that the determination ability at the time of traveling is also low.

Therefore, in the case where the awareness level of the driver is low, the driver may be prompted to perform the action of visually checking the front, the rear, the left, and the right of the vehicle before start traveling. Further, the driver's behavior may be observed on the basis of the information acquired by the occupant monitoring apparatus 10. In a case where the driver does not appropriately perform the visual checking, the control of prohibiting the starting of the vehicle may be performed.

As a result, it is possible to perform appropriate drive assistance for safe traveling.

In addition, by causing the driver to perform an action accompanying the visual checking described above, the driver can perform a stretch moving his or her neck and spine. It can be expected that such a stretch leads to an effect of promoting awakening of the driver.

### Fourth Example Embodiment

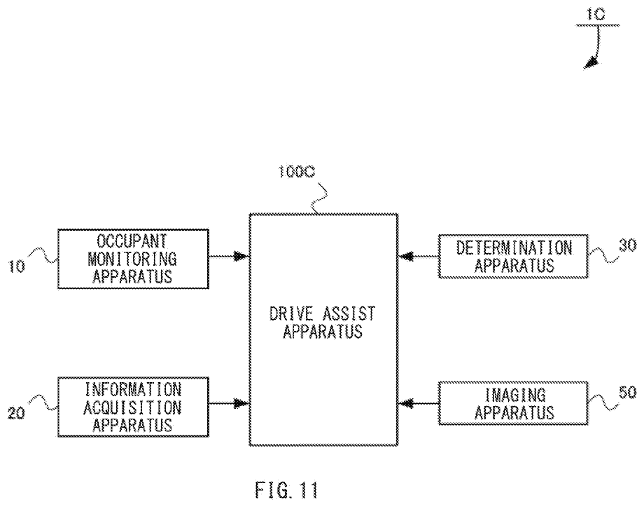

Referring to FIGS. 11 to 13, a drive assist system 1C according to a fourth example embodiment of the technology is described.

### [Configuration of Drive Assist System 1C]

As illustrated in FIG. 11, the drive assist system 1C according to the fourth example embodiment may include the occupant monitoring apparatus 10, the information acquisition apparatus 20, the determination apparatus 30, an imaging apparatus 50, and a drive assist apparatus 100C.

Note that the components denoted with the same referential signs as those in the first to the third example embodiments may serve as the same, and are therefore not described further in detail here.

The imaging apparatus 50 may capture an image of a speed sign provided in a traveling direction of the vehicle.

The image captured by the imaging apparatus 50 may be supplied to the drive assist apparatus 100C.

In a case where the awareness level of the driver is determined as being low, the drive assist apparatus 100C may acquire speed limit information from the image of the speed sign captured by the imaging apparatus 50.

Further, a drive assist control section 113C may execute a control of preventing the speed of the vehicle from being the speed limit or higher.

### [Configuration of Drive Assist Apparatus 100C]

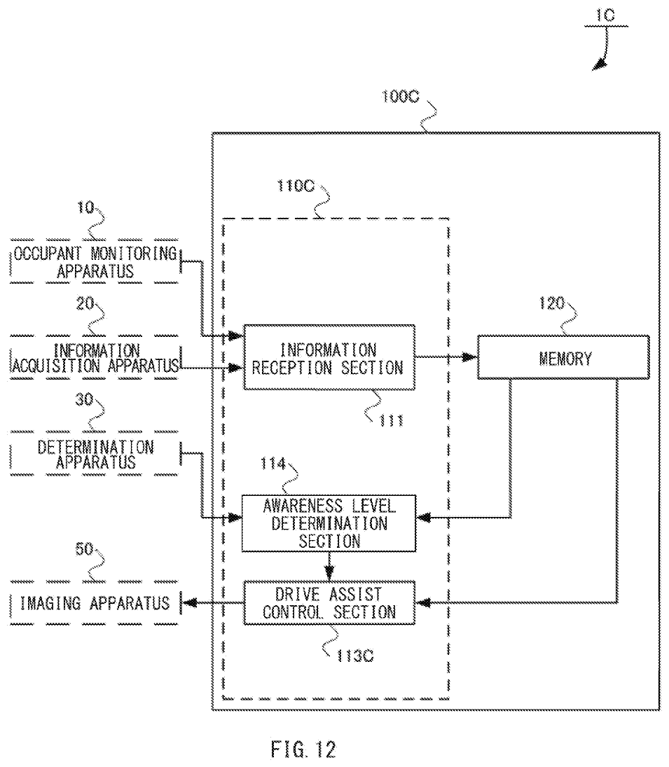

As illustrated in FIG. 12, the drive assist apparatus 100C may include a processor 110C and the memory 120. The processor 110C will be described in detail later.

### [Configuration of Processor 110C]

As illustrated in FIG. 12, the processor 110C may include the information reception section 111, the drive assist control section 113C, and the awareness level determination section 114.

In a case where the awareness level determination section 114 determines that the awareness level of the driver is low, the drive assist control section 113C may acquire the speed limit information from the image of the speed sign captured by the imaging apparatus 50.

Further, the drive assist control section 113C may execute a control of preventing the speed of the vehicle from being the speed limit or higher.

### [Process of Drive Assist Apparatus 100C]

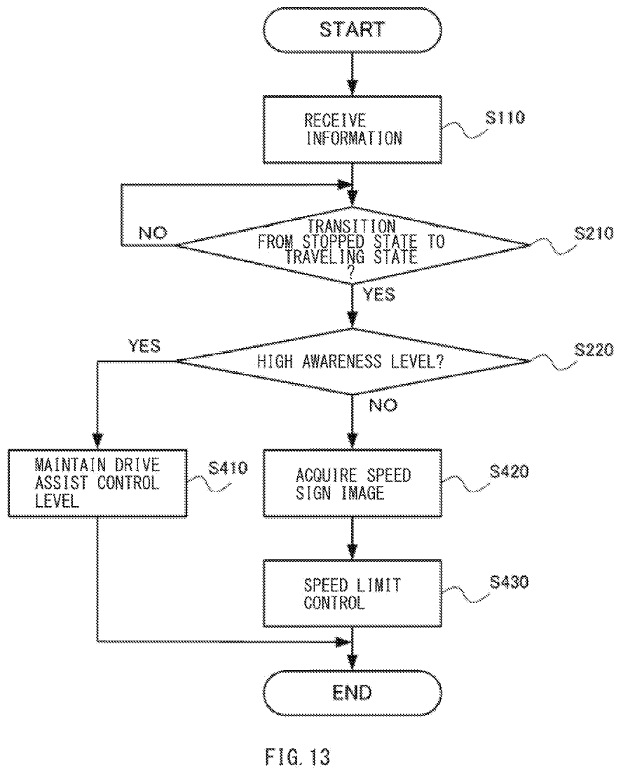

Referring to FIG. 13, a process to be performed by the drive assist apparatus 100C according to the fourth example embodiment is described.

The information reception section 111 of the drive assist apparatus 100C may receive information from the occupant monitoring apparatus 10 and information from the information acquisition apparatus 20 (step S110).

Further, the information reception section 111 may output the information received from the occupant monitoring apparatus 10 and the information acquisition apparatus 20 to the RAM in the memory 120.

The awareness level determination section 114 of the drive assist apparatus 100C may determine whether the awareness level determination section 114 has received, from the determination apparatus 30, information that the vehicle has transitioned from the stopped state to the traveling state (step S210).

Further, in a case where the awareness level determination section 114 determines that the awareness level determination section 114 has not received, from the determination apparatus 30, the information that the vehicle has transitioned from the stopped state to the traveling state (NO in step S210), the process may be caused to return to step S210, and the drive assist apparatus 100C may transition to a standby mode.

In a case where the awareness level determination section 114 determines that the awareness level determination section 114 has received, from the determination apparatus 30, the information that the vehicle has transitioned from the stopped state to the traveling state (YES in step S210), the awareness level determination section 114 may acquire, for example, information for determining the awareness level from the information stored in the RAM in the memory 120, and determine whether the awareness level of the driver is high (step S220).

In a case where the awareness level determination section 114 determines that the awareness level of the driver is high (YES in step S220), the awareness level determination section 114 may supply the information that the awareness level of the driver is high to the drive assist control section 113C.

Further, the drive assist control section 113C, which has received the information from the awareness level determination section 114, may maintain the level of the drive assist control (step S410), and end the process.

In a case where the awareness level determination section 114 determines that the awareness level of the driver is low (NO in step S220), the awareness level determination section 114 may supply the information that the awareness level of the driver is low to the drive assist control section 113C.

Further, the drive assist control section 113C, which has received the information from the awareness level determination section 114, may acquire the image of the speed sign provided in the traveling direction of the vehicle in the image captured by the imaging apparatus 50 (step S420).

Further, the drive assist control section 113C may detect the speed limit information from the acquired image of the speed sign, and execute a control of preventing the speed of the vehicle at the traveling start timing from being the speed limit or higher on the basis of the detected speed limit information (step S430).

### Example Workings and Example Effects

As described above, the drive assist system 1C according to the fourth example embodiment may include the imaging apparatus 50 that captures an image of a speed sign provided in the traveling direction of the vehicle. In a case where the awareness level determination section 114 determines that the awareness level of the driver is low, the drive assist control section 113C may acquire the speed limit information from the image of the speed sign captured by the imaging apparatus 50, and execute a control of preventing the speed of the vehicle from being the speed limit or higher.

That is, in a case of driving of the vehicle by a driver having a low awareness level, it can be considered that the determination ability at the time of traveling is also low.

Therefore, in the case where the awareness level of the driver is low, the speed limit information may be acquired from the image of the speed sign captured by the imaging apparatus 50, and a control of preventing the speed of the vehicle from being the speed limit or higher may be executed at the traveling start timing.

As a result, it is possible to perform appropriate drive assistance for safe traveling.

### Fifth Example Embodiment

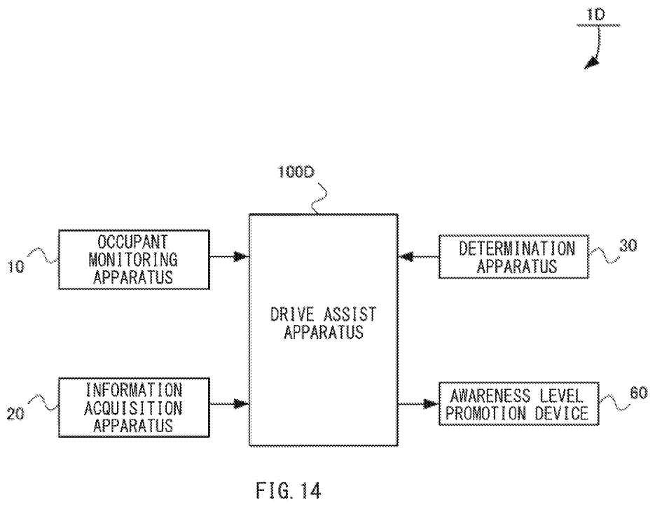

Referring to FIGS. 14 to 16, a drive assist system 1D according to a fifth example embodiment of the technology is described.

### [Configuration of Drive Assist System 1D]

As illustrated in FIG. 14, the drive assist system 1D according to the fifth example embodiment may include the occupant monitoring apparatus 10, the information acquisition apparatus 20, the determination apparatus 30, an awareness level promotion device 60, and a drive assist apparatus 100D.

Note that the components denoted with the same referential signs as those in the first to the fourth example embodiments may serve as the same, and are therefore not described further in detail here.

The awareness level promotion device 60 may increase an awareness level.

Non-limiting examples of the awareness level promotion device 60 may include a visual stimulation device, an auditory stimulation device, a somatosensory stimulation device, a gustatory stimulation device, and an olfactory stimulation device.

The visual stimulation device may be one of vehicle devices that promote awareness of the occupant. The visual stimulation device may include, for example but not limited to, a light source in the vehicle and a sunroof open and close mechanism. Non-limiting examples of the light source in the vehicle may include an interior light, a light source of a meter, a light source of a head-up display (HUD), and a light source of a monitor. For example, during nighttime, the visual stimulation device may turn on the interior light and change the luminance, the chroma, and the like of the light in accordance with a control signal. During daytime, the visual stimulation device may cause the sunroof to operate to perform operation of taking outside light into the vehicle compartment. In one example, the visual stimulation device may perform a light adjustment control with use of a glass window and thereby control the amount of outside light taken into the vehicle compartment.

The auditory stimulation device may be one of the vehicle devices that promote awareness of the occupant. The auditory stimulation device may include, for example but not limited to, an on-vehicle acoustic device. For example, the auditory stimulation device may output a warning sound, a warning message, a music, an ambient sound, a preregistered human voice, an audio sound prompting an autonomous sensory meridian response (ASMR), and/or any other suitable sound.

The somatosensory stimulation device may be one of the vehicle devices that promote awareness of the occupant. The somatosensory stimulation device may include, for example but not limited to, a vibration mechanism that vibrates a seating surface or a back surface of a seat and a vibration mechanism that vibrates a headrest. The somatosensory stimulation device may also be configured to output an image or an audio sound regarding how to perform massage for recovery from fatigue of each target part.

The olfactory stimulation device may be one of the vehicle devices that promote awareness of the occupant. The olfactory stimulation device may include, for example but not limited to, an aroma generation device. For example, the olfactory stimulation device may generate a scent that promotes awareness of a fatigue person, such as a scent of mint or an aroma oil. For example, the olfactory stimulation device may generate: a scent that increases concentration, such as a scent of rosemary, bergamot, geranium, peppermint, eucalyptus, or lemon; a scent that promotes relaxation such as a scent of lavender, chamomile, juniper, neroli, yuzu, ylang-ylang, or rosewood; or a scent that promotes recovery from fatigue such as a scent of sweet marjoram, rosemary, ginger, juniper, marjoram, lavender, or chamomile.

In a case where the awareness level of the driver is determined as being low, the drive assist apparatus 100D may take a measure to increase the awareness level before the vehicle starts traveling, and execute a control of prohibiting starting of the vehicle until the measure is completed.

### [Configuration of Drive Assist Apparatus 100D]

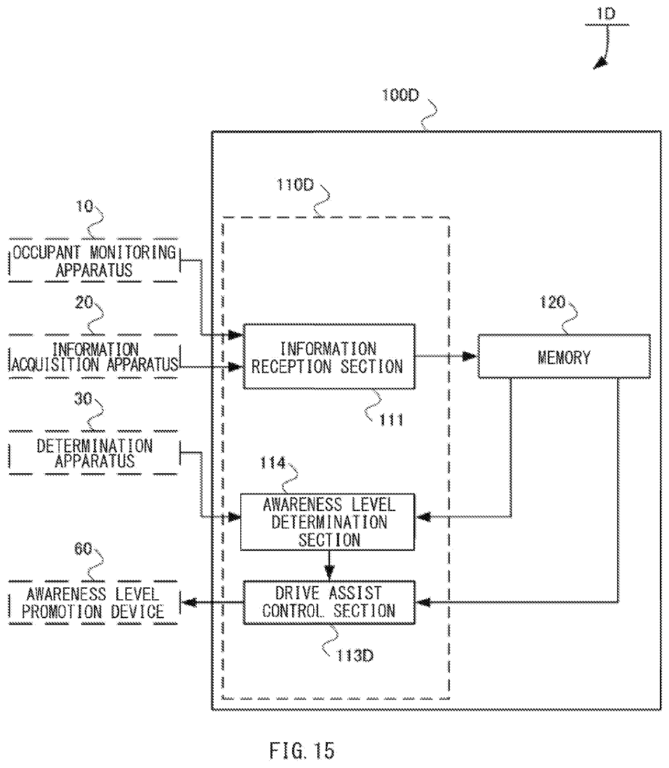

As illustrated in FIG. 15, the drive assist apparatus 100D may include a processor 110D and the memory 120. The processor 110D will be described in detail later. [Configuration of Processor 110D]

As illustrated in FIG. 15, the processor 110D may include the information reception section 111, a drive assist control section 113D, and the awareness level determination section 114.

In a case where the awareness level determination section 114 determines that the awareness level of the driver is low, the drive assist control section 113D may take the measure to increase the awareness level of the driver by the awareness level promotion device 60 before the vehicle starts traveling, and execute the control of prohibiting the starting of the vehicle until the measure is completed.

### [Process of Drive Assist Apparatus 100D]

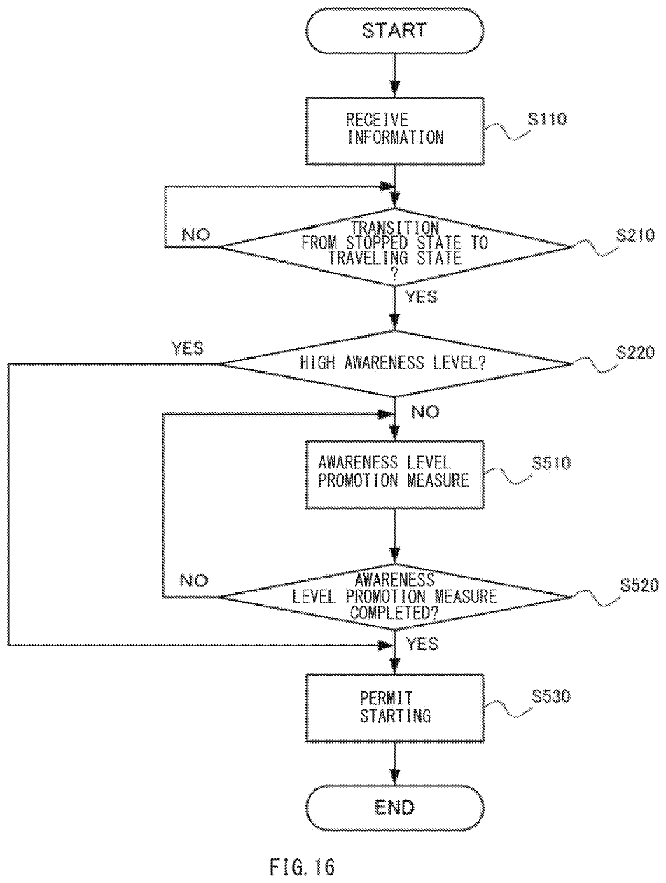

Referring to FIG. 16, a process to be performed by the drive assist apparatus 100D according to the fifth example embodiment is described.

The information reception section 111 of the drive assist apparatus 100D may receive information from the occupant monitoring apparatus 10 and information from the information acquisition apparatus 20 (step S110).

Further, the information reception section 111 may output the information received from the occupant monitoring apparatus 10 and the information acquisition apparatus 20 to the RAM in the memory 120.

The awareness level determination section 114 of the drive assist apparatus 100D may determine whether the awareness level determination section 114 has received, from the determination apparatus 30, information that the vehicle has transitioned from the stopped state to the traveling state (step S210).

Further, in a case where the awareness level determination section 114 determines that the awareness level determination section 114 has not received, from the determination apparatus 30, the information that the vehicle has transitioned from the stopped state to the traveling state (NO in step S210), the process may be caused to return to step S210, and the drive assist apparatus 100D may transition to a standby mode.

In a case where the awareness level determination section 114 determines that the awareness level determination section 114 has received, from the determination apparatus 30, the information that the vehicle has transitioned from the stopped state to the traveling state (YES in step S210), the awareness level determination section 114 may acquire, for example, information for determining the awareness level from the information stored in the RAM in the memory 120, and determine whether the awareness level of the driver is high (step S220).

In a case where the awareness level determination section 114 determines that the awareness level of the driver is high (YES in step S220), the awareness level determination section 114 may supply the information that the awareness level of the driver is high to the drive assist control section 113D.

Further, the drive assist control section 113D, which has received the information from the awareness level determination section 114, may permit the starting of the vehicle (step S530).

In a case where the awareness level determination section 114 determines that the awareness level of the driver is low (NO in step S220), the awareness level determination section 114 may supply the information that the awareness level of the driver is low to the drive assist control section 113D.

Further, the drive assist control section 113D, which has received the information from the awareness level determination section 114, may take the measure to increase the awareness level by the awareness level promotion device (step S510).

Further, the drive assist control section 113D may determine whether the measure to increase the awareness level by the awareness level promotion device 60 is completed (step S520). In a case where the measure to increase the awareness level by the awareness level promotion device 60 is determined as not being completed (NO in step S520), the process may be caused to return to step S510.

In a case where the measure to increase the awareness level by the awareness level promotion device 60 is determined as being completed (YES in step S520), the drive assist control section 113D may permit the starting of the vehicle (step S530).

### Example Workings and Example Effects

As described above, in the drive assist system 1D according to the fifth example embodiment, in a case where the awareness level determination section 114 determines that the awareness level of the driver is low, the drive assist control section 113D may take the measure to increase the awareness level by the awareness level promotion device 60, and execute a control of prohibiting the starting of the vehicle.

That is, in a case of driving of the vehicle by a driver having a low awareness level, it can be considered that the determination ability at the time of traveling is also low.

Therefore, in the case where the awareness level of the driver is low, the measure to increase the awareness level by the awareness level promotion device 60 may be taken before the vehicle starts traveling, and a control of prohibiting the starting of the vehicle may be executed until the measure is completed.

As a result, it is possible to perform appropriate drive assistance for safe traveling.

### Sixth Example Embodiment

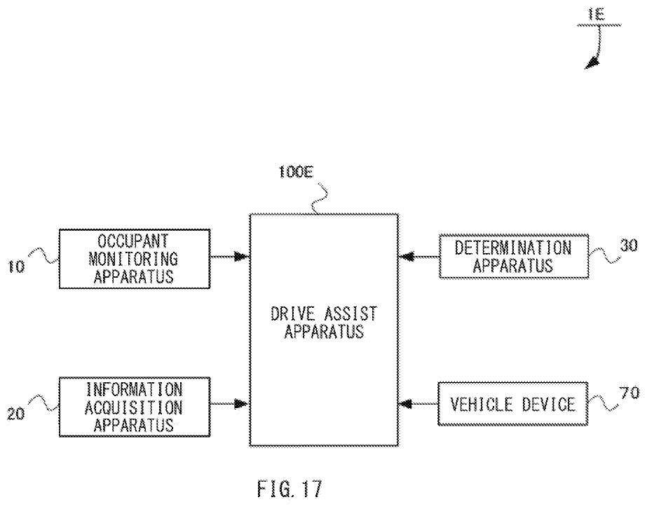

Referring to FIGS. 17 to 19, a drive assist system 1E according to a sixth example embodiment of the technology is described.

### [Configuration of Drive Assist System 1E]

As illustrated in FIG. 17, the drive assist system 1E according to the sixth example embodiment may include the occupant monitoring apparatus 10, the information acquisition apparatus 20, the determination apparatus 30, a vehicle device 70, and a drive assist apparatus 100E.

Note that the components denoted with the same referential signs as those in the first to the fifth example embodiments may serve as the same, and are therefore not described further in detail here.

Non-limiting examples of the vehicle device 70 may include an illumination device, an acoustic device, an air conditioning device, and a seating device.

The vehicle device 70 may allow inside of the vehicle compartment to be a vehicle compartment environment appropriate for sleeping, on the basis of a control signal supplied from the drive assist apparatus 100E.

In a case where the awareness level of the driver is determined as being low, the drive assist apparatus 100E may prompt the driver to sleep and allow the inside of the vehicle compartment to be the vehicle compartment environment appropriate for sleeping. The drive assist apparatus 100E may also perform a control of prohibiting starting of the vehicle until the driver takes a necessary sleep.

### [Configuration of Drive Assist Apparatus 100E]

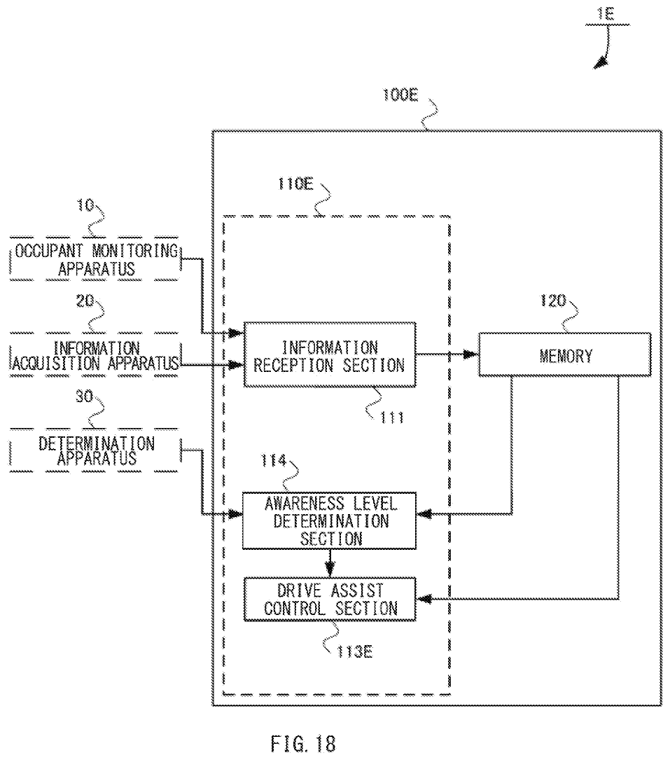

As illustrated in FIG. 18, the drive assist apparatus 100E may include a processor 110E and the memory 120. The processor 110E will be described in detail later.

### [Configuration of Processor 110E]

As illustrated in FIG. 18, the processor 110E may include the information reception section 111, a drive assist control section 113E, and the awareness level determination section 114.

In a case where the awareness level determination section 114 determines that the awareness level of the driver is low, the drive assist control section 113E may prompt the driver to sleep and allow the inside of the vehicle compartment to be the vehicle compartment environment appropriate for sleeping.

Further, the drive assist control section 113E may perform a control of prohibiting the starting of the vehicle until the driver takes a necessary sleep.

### [Process of Drive Assist Apparatus 100E]

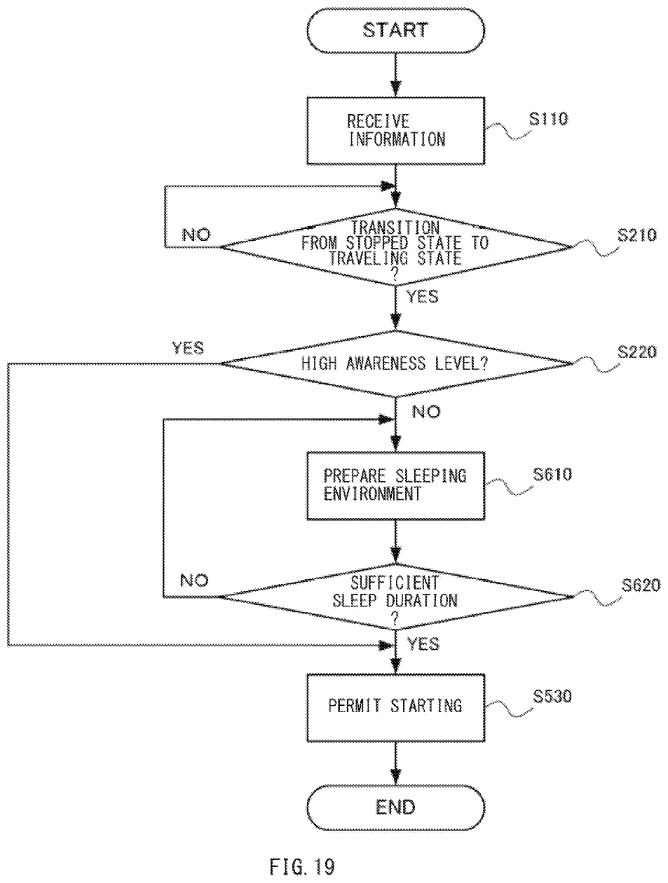

Referring to FIG. 19, a process to be performed by the drive assist apparatus 100E according to the sixth example embodiment is described.

The information reception section 111 of the drive assist apparatus 100E may receive information from the occupant monitoring apparatus 10 and information from the information acquisition apparatus 20 (step S110).

Further, the information reception section 111 may output the information received from the occupant monitoring apparatus 10 and the information acquisition apparatus 20 to the RAM in the memory 120.

The awareness level determination section 114 of the drive assist apparatus 100E may determine whether the awareness level determination section 114 has received, from the determination apparatus 30, information that the vehicle has transitioned from the stopped state to the traveling state (step S210).

Further, in a case where the awareness level determination section 114 determines that the awareness level determination section 114 has not received, from the determination apparatus 30, the information that the vehicle has transitioned from the stopped state to the traveling state (NO in step S210), the process may be caused to return to step S210, and the drive assist apparatus 100E may transition to a standby mode.

In a case where the awareness level determination section 114 determines that the awareness level determination section 114 has received, from the determination apparatus 30, the information that the vehicle has transitioned from the stopped state to the traveling state (YES in step S210), the awareness level determination section 114 may acquire, for example, information for determining the awareness level from the information stored in the RAM in the memory 120, and determine whether the awareness level of the driver is high (step S220).

In a case where the awareness level determination section 114 determines that the awareness level of the driver is high (YES in step S220), the awareness level determination section 114 may supply the information that the awareness level of the driver is high to the drive assist control section 113E.

The drive assist control section 113E, which has received the information from the awareness level determination section 114, may permit the starting of the vehicle (step S530).

In a case where the awareness level determination section 114 determines that the awareness level of the driver is low (NO in step S220), the awareness level determination section 114 may supply the information that the awareness level of the driver is low to the drive assist control section 113E.

Further, the drive assist control section 113E, which has received the information from the awareness level determination section 114, may supply a control signal to the vehicle device 70, and execute a control of allowing the inside of the vehicle compartment to be the vehicle compartment environment appropriate for sleeping (step S610).

For example, during the nighttime, the drive assist control section 113E may make the illumination appropriately darker, play a music that promotes sleeping, make the temperature of the vehicle compartment appropriate, and adjust an angle of a back seat of the seating device to be appropriate for sleeping.

The drive assist control section 113E may determine whether the driver has had a sufficient sleep on the basis of the information from the occupant monitoring apparatus 10 and the information from the information acquisition apparatus 20 (step S620). In a case where the driver is determined as not having had a sufficient sleep yet (NO in step S620), the drive assist control section 113E may cause the process to return to step S610.

In a case where the driver is determined as having had a sufficient sleep (YES in step S620), the drive assist control section 113E may permit the starting of the vehicle (step S530).

### Example Workings and Example Effects

As described above, in the drive assist system 1E according to the sixth example embodiment, in a case where the awareness level determination section 114 determines the awareness level of the driver is low, the drive assist control section 113E may prompt the driver to sleep and allow the inside of the vehicle compartment to be the vehicle compartment environment appropriate for sleeping. Further, the drive assist control section 113E may perform the control of prohibiting the starting of the vehicle until the driver takes a necessary sleep.

That is, in a case of driving of the vehicle by a driver having a low awareness level, it can be considered that the determination ability at the time of traveling is also low.

Therefore, in the case where the awareness level of the driver is low, the driver may be prompted to take a sufficient sleep, and the inside of the vehicle compartment may be allowed to be a vehicle compartment environment appropriate for sleeping before the vehicle starts traveling. Further, the control of prohibiting the starting of the vehicle may be executed until the driver takes a necessary sleep.

As a result, it is possible to perform appropriate drive assistance for safe traveling.

### Modifications

For example, the above example embodiments each have been described referring to a configuration in which the drive assist apparatus 100, 100A, 100B, 100C, 100D, or 100E is provided in the corresponding one of the drive assist systems 1, 1A, 1B, 1C, 1D, and 1E. However, this is non-limiting. One example embodiment may have a configuration in which the information acquired by the occupant monitoring apparatus 10 and the information acquired by the information acquisition apparatus 20 are transferred to a server coupled to the vehicle, and the control process performed by the drive assist apparatus 100, 100A, 100B, 100C, 100D, or 100E may be performed in the server.

This configuration may allow for quick processing of more information to increase the awareness level of the driver. As a result, it is possible to reduce a possibility of occurrence of an accident.

The first example embodiment has been described referring to an example configuration in which the sleep determination section 112 receives, from the determination apparatus 30, the information that the vehicle has transitioned from the stopped state to the traveling state, and determines whether the vehicle has transitioned from the stopped state to the traveling state. The second example embodiment has been described referring to an example configuration in which the awareness level determination section 114 receives, from the determination apparatus 30, the information that the vehicle has transitioned from the stopped state to the traveling state, and determines whether the vehicle has transitioned from the stopped state to the traveling state. However, these are non-limiting. In one example, the drive assist control section 113 or 113A may perform the above-described processes of information reception and determination.

Note that the drive assist system according to an embodiment of the technology may be implementable by: recording the process to be performed by the processor 110, 110A, 110B, 110C, 110D, or 110E in a recording medium readable by a computer system; and causing the processor 110, 110A, 110B, 110C, 110D, or 110E to read the program recorded in the recording medium to execute the program. The above-mentioned computer system encompasses an operating system (OS) and hardware such as a peripheral apparatus.

Further, two or more processors 110, 110A, 110B, 110C, 110D, or 110E may be provided. Further, two or more memories 120 may be provided.

In a case where the world wide web (WWW) system is used, the above-mentioned “computer system” encompasses a website provision environment or a website display environment. Further, the above-mentioned program may be transferred from the computer system having a storage device or the like holding the program to another computer system via a transfer medium or a transfer wave in the transfer medium. As used herein, the term “transfer medium” that transfers the program refers to a medium configured to transfer information, for example, a network (a communication network) such as the Internet or a communication line (a communication wire) such as a telephone line.

Moreover, the above-mentioned program may implement a portion of the above-described process to be performed by the drive assist system according to an embodiment of the technology.

Moreover, a combination of the above-mentioned program and another program pre-recorded in the computer system may implement the above-described process to be performed by the drive assist system according to an embodiment of the technology. That is, the above-mentioned program may be a differential file (a differential program).

Although some embodiments of the technology have been described in the foregoing by way of example with reference to the accompanying drawings, the technology is by no means limited to the embodiments described above. It should be appreciated that modifications and alterations may be made by persons skilled in the art without departing from the scope as defined by the appended claims. The technology is intended to include such modifications and alterations in so far as they fall within the scope of the appended claims or the equivalents thereof.

According to one or more example embodiments of the technology, the sleep state of the occupant, including the driver, inside the vehicle may be determined at the driving start timing of the vehicle. Accordingly, the one or more example embodiments of the technology achieve an effect that it is possible to perform appropriate drive assistance for safe traveling.

Each of the processors 110, 110A, 110B, 110C, 110D, and 110E, the information reception section 111, the sleep determination section 112, the drive assist control section 113, 113A, 113B, 113C, 113D, and 113E, and the awareness level determination section 114 illustrated in FIGS. 2, 6, 9, 12, 15, and 18 is implementable by circuitry including at least one semiconductor integrated circuit such as at least one processor (e.g., a central processing unit (CPU)), at least one application specific integrated circuit (ASIC), and/or at least one field programmable gate array (FPGA). At least one processor is configurable, by reading instructions from at least one machine readable non-transitory tangible medium, to perform all or a part of functions of each of the processors 110, 110A, 110B, 110C, 110D, and 110E, the information reception section 111, the sleep determination section 112, the drive assist control section 113, 113A, 113B, 113C, 113D, and 113E, and the awareness level determination section 114 illustrated in FIGS. 2, 6, 9, 12, 15, and 18. Such a medium may take many forms, including, but not limited to, any type of magnetic medium such as a hard disk, any type of optical medium such as a CD and a DVD, any type of semiconductor memory (i.e., semiconductor circuit) such as a volatile memory and a non-volatile memory. The volatile memory may include a DRAM and a SRAM, and the nonvolatile memory may include a ROM and a NVRAM. The ASIC is an integrated circuit (IC) customized to perform, and the FPGA is an integrated circuit designed to be configured after manufacturing in order to perform, all or a part of the functions of each of the processors 110, 110A, 110B, 110C, 110D, and 110E, the information reception section 111, the sleep determination section 112, the drive assist control section 113, 113A, 113B, 113C, 113D, and 113E, and the awareness level determination section 114 illustrated in FIGS. 2, 6, 9, 12, 15, and 18.

1. A drive assist system comprising to be applied to the vehicle, the drive assist system comprising:
an occupant monitoring apparatus configured to monitor a physical state of an occupant of the vehicle, the occupant including a driver who drives the vehicle;
an information acquisition apparatus configured to acquire information including biometric information of the occupant when inside and outside the vehicle;
a determination apparatus configured to determine a traveling state and a stopped state of the vehicle; and
a drive assist apparatus configured to determine a sleep state of the occupant on a basis of monitoring information acquired by the occupant monitoring apparatus, acquisition information acquired by the information acquisition apparatus, or both, and execute a drive assist control for securing safe traveling of the vehicle, the sleep state including sleep duration and quality of sleep,
the drive assist apparatus including
one or more processors, and
one or more memories coupled to the one or more processors to communicate with the one or more processors,
the one or more processors being configured to appropriately change a warning timing or a drive assist control intervention timing in accordance with the sleep state of the driver at least from a previous day, upon receiving information, from the determination apparatus, that the vehicle transitions from the stopped state to the traveling state, the warning timing being a timing of giving a warning that the vehicle is approaching another vehicle, an object, a human, or an animal, the drive assist control intervention timing being a timing of intervention by the drive assist control for avoiding contact between the vehicle and the other vehicle, the object, the human, or the animal.

2. The drive assist system according to claim 1, wherein the one or more processors are configured to refrain from changing the warning timing or the drive assist control intervention timing in a case where accumulated sleep duration of the driver at least from the previous day is a predetermined time or more.

3. The drive assist system according to claim 2, wherein the one or more processors are configured to set the predetermined time from the accumulated sleep duration of each day measured for each the driver, on the basis of the monitoring information, the acquisition information, or both.

4. The drive assist system according to claim 1, wherein the one or more processors are configured to refrain from changing the warning timing or the drive assist control intervention timing in a case where the quality of sleep of the driver is a predetermined level or higher.

5. The drive assist system according to claim 1, wherein
the one or more processors are configured to
determine an awareness level of the driver on the basis of the monitoring information, the acquisition information, or both, and
in a case where the awareness level of the driver is determined as being low, increase a level of the drive assist control for securing the safe traveling of the vehicle.

6. The drive assist system according to claim 5, wherein the one or more processors are configured to determine the awareness level of the driver from a sleep start time and the sleep duration of the driver at least of a previous night on the basis of the monitoring information, the acquisition information, or both.

7. The drive assist system according to claim 5, wherein the one or more processors are configured to determine the awareness level of the driver from variation in a brain wave of the driver on a basis of the acquisition information.

8. The drive assist system according to claim 5, wherein
the one or more processors are configured to
in a case where the awareness level of the driver is determined as being low, prompt the driver to perform an action of visually checking front, rear, left, and right of the vehicle before the vehicle starts traveling,
observe behavior of the driver on a basis of the monitoring information acquired by the occupant monitoring apparatus, and
in a case where the driver does not appropriately perform the action of visually checking, perform a control of prohibiting starting of the vehicle.

9. The drive assist system according to claim 5, further comprising
an imaging apparatus configured to capture an image of a speed sign provided in a traveling direction of the vehicle, wherein
the one or more processors are configured to, in the case where the awareness level of the driver is determined as being low, acquire speed limit information from the image of the speed sign captured by the imaging apparatus, and perform a control of preventing a speed of the vehicle from being a speed limit or higher before the vehicle starts traveling.

10. The drive assist system according to claim 5, wherein the one or more processors are configured to, in the case where the awareness level of the driver is determined as being low, take a measure to increase the awareness level before the vehicle starts traveling, and perform a control of prohibiting starting of the vehicle until the measure is completed.

11. The drive assist system according to claim 5, wherein
the one or more processors are configured to
in the case where the awareness level of the driver is determined as being low, prompt the driver to take a sleep and allow inside of a vehicle compartment of the vehicle to be a vehicle compartment environment appropriate for sleeping before the vehicle starts traveling, and
perform a control of prohibiting starting of the vehicle until the driver takes a necessary sleep.
